(19) United States
(12) Patent Application Publication
(10) Pub. No.: US 2018/0369342 A1
(43) Pub. Date: Dec. 27, 2018

(54) CARDIAC METABOLIC EFFECT OF LANTUS

(71) Applicant: Sanofi-Aventis Deutschland GmbH, Frankfurt am Main (DE)

(72) Inventors: Paulus Wohlfahrt, FRANKFURT am Main (DE); Norbert Tennagels, FRANKFURT am Main (DE); Gary Lopaschuk, Edmonton (CA)

(21) Appl. No.: 16/064,313

(22) PCT Filed: Dec. 21, 2016
(86) PCT No.: PCT/EP2016/082166

§ 371 Date: Jun. 20, 2018

(30) Foreign Application Priority Data
Dec. 23, 2015 (EP) 15202428.7

(51) Int. Cl. A61K 38/28; A61P 3/08; A61P 9/08; A61P 9/02; A61P 9/10

(52) U.S. Cl. CPC A61K 38/28; A61P 3/08; A61P 9/10; A61P 9/02; A61P 9/08

(57) ABSTRACT

The present invention relates to a insulin or insulin analogue for use in increasing cardiac efficiency or cardiac stroke volume in a patient at risk of developing or suffering from diabetes, as well as for use in preventing and/or treating cardiovascular diseases in patients at risk of developing or suffering from diabetes, to methods of preventing delaying, and/or treating cardiovascular diseases in patients at risk of developing or suffering from diabetes, to pharmaceutical compositions for use in increasing cardiac efficiency or cardiac stroke volume or for use in preventing and/or treating cardiovascular diseases comprising an insulin analogue, and to methods of identifying a patient who may benefit from treatment with an insulin analogue.

The present invention relates to a insulin or insulin analogue for use in increasing cardiac efficiency or cardiac stroke volume in a patient at risk of developing or suffering from diabetes, as well as for use in preventing and/or treating cardiovascular diseases in patients at risk of developing or suffering from diabetes, to methods of preventing delaying, and/or treating cardiovascular diseases in patients at risk of developing or suffering from diabetes, to pharmaceutical compositions for use in increasing cardiac efficiency or cardiac stroke volume or for use in preventing and/or treating cardiovascular diseases comprising an insulin analogue, and to methods of identifying a patient who may benefit from treatment with an insulin analogue.

### BACKGROUND

Insulin therapy of diabetic patients is characterized by a high need for keeping the insulin drug release within very strict levels as the therapeutic window is narrow, and the adverse effects of hyperinsulinemia can potentially be life threatening. Numerous insulin preparations have been commercialized, with different action profiles to suit specific needs of the diabetic population. Fast acting insulin analogs are administered just before meals, in order to control the peak in plasma glucose following food ingestion, whereas long acting insulin analogs are typically given once or twice a day to provide a steady basal insulin level. Current standard basal insulin therapy consists of daily or twice daily administrations of long acting basal insulins such as NPH insulin, insulin glargine or insulin detemir, and degludec Diabetes is associated with a large variety of serious long-term consequences which include cardiovascular disease and associated diseases or disorders.

The heart must continuously generate large amounts of adenosine triphosphate (ATP) in order to maintain contractile function. The continuous synthesis of ATP in the heart is primarily met by the metabolism of fatty acids and carbohydrates¹⁻⁴. In the adult heart over 90% of the ATP supply is generated from mitochondrial oxidative phosphorylation, with the remainder originating from glycolysis. The majority of this mitochondrial ATP production normally originates from fatty acid β-oxidation.¹⁻⁴ However, the heart can rapidly switch to other fuel sources (such as carbohydrate oxidation, ketone oxidation, and amino acid metabolism) in response to a number of factors, including: i) contractile demand, ii) nutritional status, iii) hormonal influences, iv) oxygen supply, v) transcriptional/translational and post-translational control of energy metabolic pathways, and/or vi) the presence of underlying cardiac disease(s).¹⁻⁴

In obese and diabetic individuals, the heart switches its energy substrate preference from glucose metabolism to fatty acid metabolism. In addition, in insulin resistance and diabetes, the heart also becomes insulin resistant. Insulin resistance at the level of the heart also occurs in the failing heart. This cardiac insulin resistance contributes to severity of heart failure.¹⁻⁴ Decreased insulin stimulation of glucose oxidation in the failing heart contributes to both an impairment of heart efficiency.¹⁻⁴ This decrease in cardiac efficiency can contribute to cardiac dysfunction.

In view of the above, it would be desirable to provide patients with a long acting preparations of insulin which do not only provide systemic metabolic control of hyperglycemia but which also reduce the risk of diabetes associated complications, including the development of heart failure and related forms of cardiac dysfunction. There is thus, an urgent need for a therapeutic approach increasing glucose oxidation to improve the function of the failing heart. Further, it is desirable to provide a therapeutic approach for insulin resistance and cardiac diastolic dysfunction by improving cardiac energy metabolism.

### SUMMARY OF THE INVENTION

In a first aspect, the present invention provides an insulin analogue for use in increasing cardiac output or cardiac stroke volume in a patient at risk of developing or suffering from diabetes.

In a second aspect, the present invention provides an insulin analogue for use in preventing, delaying and/or treating cardiovascular diseases in patients at risk of developing or suffering from diabetes.

In a third aspect, the present invention relates to a method of preventing, delaying and/or treating cardiovascular diseases in patients at risk of developing or suffering from diabetes comprising administering a therapeutically effective amount of an insulin analogue.

In a fourth aspect, the present invention relates to a pharmaceutical composition for use in increasing cardiac efficiency or cardiac stroke volume or for use in preventing, delaying and/or treating cardiovascular diseases comprising an insulin analogue and at least one pharmaceutical acceptable carrier, adjuvant and/or excipient.

In a fifth aspect, the present invention provides a method of identifying a patient or a group of patients who may benefit from treatment with an insulin analogue, wherein said patient or group of patients is at risk of developing or suffers from diabetes, comprising the step of determining whether said patient suffers from decreased cardiac output or decreased cardiac stroke volume or from a cardiovascular disease.

In a sixth aspect, the present invention relates to a method of identifying a patient or a group of patients who will benefit from treatment with an insulin analogue, wherein said patient or group of patients is at risk of developing or suffers from diabetes and exhibits increased fatty acid oxidation and/or reduced glucose oxidation in the heart.

In a seventh aspect, the present invention relates to an insulin analogue for use in preventing, delaying and/or treating a cardiovascular disease in a patient at risk of developing or suffering from diabetes, wherein said patient exhibits increased fatty acid oxidation and/or reduced glucose oxidation in the heart.

### LIST OF FIGURES

FIG. 1: Experimental design for acute treatment with insulin, M1 lantus, and degludec.

FIG. 2: Effect of acute treatment with insulin, degludec, and lantus on glucose oxidation in C57bl/6 and db/db mouse hearts (A, C), and palmitate oxidation in C57bl/6 and db/db mouse hearts (B, D).

FIG. 3: Effect of acute treatment with insulin, degludec, and lantus on cardiac efficiency in C57bl/6 (A) and in db/db (B) mouse hearts.

FIG. 4: Lantus acutely stimulates glucose oxidation, decreases palmitate oxidation, and improves cardiac efficiency in 10 wk old C57bl/6 mouse hearts.

FIG. 5: Lantus acutely stimulates glucose oxidation and decreases palmitate oxidation in 10 wk old db/db mouse hearts.

FIG. 6: Effect of acute treatment with insulin, degludec and lantus on % ATP production in C57bl/6 (A) and db/db (B) mouse hearts.

FIG. 7: Effect of acute treatment with insulin, degludec, and lantus on cardiac Akt phosphorylation in C57bl/6 and in db/db mouse hearts.

FIG. 8: Experimental design for chronic treatment with NPH insulin, degludec and lantus

FIG. 9: Chronic treatment with long acting insulin improves whole body glucose tolerance in db/db mice.

FIG. 10: Chronic treatment with lantus improves in vivo cardiac function in db/db mice. A, C: cardiac output; B, D: Stroke volume

FIG. 11: Effect of chronic treatment with long acting insulin on in vivo cardiac function in db/db mice. A: Corr LV Mass; B: E/A, C: E′/A′; D: E/E′

FIG. 12: Effect of chronic treatment with long acting insulin on in vivo cardiac function and heart mass in db/db mice. A: Tei Index; B: % EF; C: IVRT; D: Wet heart weight (mg); E: Wet heart weight/Tibia length

### LIST OF REFERENCES

1. Lopaschuk G D, Ussher J R, Folmes C D, Jaswal J S, Stanley W C. Myocardial fatty acid metabolism in health and disease. Physiol Rev. 2010; 90(1):207-258.

2. Neely J M, H E. Relationship between carbohydrate metabolism and energy balance of heart muscle. Ann rev Physiol. 1974; 36:413-459.

3. Saddik M, Lopaschuk G D. Myocardial triglyceride turnover and contribution to energy substrate utilization in isolated working rat hearts. J Biol Chem. 1991; 266(13):8162-8170.

4. Stanley W C, Lopaschuk G D, Hall J L, McCormack J G. Regulation of myocardial carbohydrate metabolism under normal and ischaemic conditions. Potential for pharmacological interventions. Cardiovasc Res. 1997; 33(2):243-257.

5. Yancy C W, et al. ACCF/AHA guideline for the management of heart failure: a report of the American College of Cardiology Foundation/American Heart Association Task Force on Practice Guidelines. Circulation. 2013:128:e240-e327.

6. McMurray J J V et al. ESC Guidelines for the diagnosis and treatment of acute and chronic heart failure 2012European Heart Journal 2012:33, 1787-1847.

7. Rider O J, and Tyler D J. Clinical Implications of Cardiac Hyperpolarized Magnetic Resonance Imaging. Journal of Cardiovascular Magnetic Resonance 2013:15:93

8. Salamanca-Cardona L, and Keshari K R. ¹³C-labeled biochemical probes for the study of cancer metabolism with dynamic nuclear polarization-enhanced magnetic resonance imaging. Cancer & Metabolism 2015:3:9.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

Amino acids are organic compounds composed of amine (—NH2) and carboxylic acid (—COOH) functional groups, along with a side-chain specific to each amino acid. Typically, amino acids are classified by the properties of their side-chain into four groups: the side-chain can make an amino acid a weak acid or a weak base, a hydrophile if the side-chain is polar or a hydrophobe if it is nonpolar.

In the context of the different aspects of present invention, the term “peptide” refers to a short polymer of amino acids linked by peptide bonds. It has the same chemical (peptide) bonds as proteins, but is commonly shorter in length. The shortest peptide is a dipeptide, consisting of two amino acids joined by a single peptide bond. There can also be a tripeptide, tetrapeptide, pentapeptide, etc. Preferably, the peptide has a length of up to 8, 10, 12, 15, 18 or 20 amino acids. A peptide has an amino end and a carboxyl end, unless it is a cyclic peptide.

In the context of the different aspects of present invention, the term “polypeptide” refers to a single linear chain of amino acids bonded together by peptide bonds and preferably comprises at least about 21 amino acids. A polypeptide can be one chain of a protein that is composed of more than one chain or it can be the protein itself if the protein is composed of one chain.

In the context of the different aspects of present invention, the term “protein” refers to a molecule comprising one or more polypeptides that resume a secondary and tertiary structure and additionally refers to a protein that is made up of several polypeptides, i.e. several subunits, forming quaternary structures. In the context of present invention, the primary structure of a protein or polypeptide is the sequence of amino acids in the polypeptide chain. The secondary structure in a protein is the general three-dimensional form of local segments of the protein. It does not, however, describe specific atomic positions in three-dimensional space, which are considered to be tertiary structure. In proteins, the secondary structure is defined by patterns of hydrogen bonds between backbone amide and carboxyl groups. The tertiary structure of a protein is the three-dimensional structure of the protein determined by the atomic coordinates. The quaternary structure is the arrangement of multiple folded or coiled protein or polypeptide molecules molecules in a multi-subunit complex. The terms “amino acid chain” and “polypeptide chain” are used synonymously in the context of present invention.

Proteins and polypeptide of the present invention (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility. Chemical modifications applicable to the variants usable in the present invention include without limitation: PEGylation, glycosylation of non-glycosylated parent polypeptides, or the modification of the glycosylation pattern present in the parent polypeptide. The protein may also have non-peptide groups attached, such as e.g. prosthetic groups or cofactors.

As used herein, the term “variant” is to be understood as a polypeptide or protein which differs in comparison to the polypeptide or protein from which it is derived by one or more changes in its length or sequence. The polypeptide or protein from which a polypeptide variant or protein variant is derived is also known as the parent polypeptide or protein. The term “variant” comprises “fragments”, “analogues” and “derivatives” of the parent molecule. Typically, “fragments” are smaller in length or size than the parent molecule, whilst “derivatives” exhibit one or more differences in their sequence in comparison to the parent molecule. A variant may be constructed artificially, preferably by gene-technological means whilst the parent polypeptide or protein is a wild-type polypeptide or protein. Also encompassed are modified molecules such as but not limited to post-translationally modified proteins (e.g. glycosylated, biotinylated, phosphorylated, ubiquitinated, palmitoylated, or proteolytically cleaved proteins). However, also naturally occurring variants are to be understood to be encompassed by the term “variant” as used herein.

A “variant” as used herein, can be characterized by a certain degree of sequence identity to the parent polypeptide or parent protein from which it is derived. More precisely, a protein variant in the context of the present invention exhibits at least 80% sequence identity to its parent polypeptide. The term “at least 80% sequence identity” is used throughout the specification with regard to polypeptide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Preferably, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide.

Proteins which differ through substitution of at least one naturally occurring amino acid residue by other amino acid residues and/or addition and/or deletion of at least one amino acid residue from the corresponding, otherwise identical naturally occurring protein are referred to as “analogues” of proteins. It is also possible in this connection for the added and/or replaced amino acid residues to be ones which do not occur naturally. An example of a protein analogue is insulin Glargine, wherein in human insulin at position 21 of the A-chain the Asn residue is replaced with a Gly residue. Additionally at position 30 of the B-chain two Arg residues are added. Metabolite Glargine-M1 is based on insulin Glargine but lacks the two additional Arg residues added.

Proteins which are obtained by chemical modification of certain amino acid residues of initial proteins are referred to as “derivatives” of proteins. The chemical modification may consist for example of addition of one or more particular chemical groups to one or more amino acids. Semi-conservative and especially conservative amino acid substitutions, wherein an amino acid is substituted with a chemically related amino acid, are preferred. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure. Proteins which are obtained by chemical modification of certain amino acid residues of initial proteins are referred to as “derivatives” of proteins. The chemical modification may consist for example of addition of one or more particular chemical groups to one or more amino acids. An example of a protein derivative is insulin Degludec which differs from normal human insulin in that the last amino acid of the B-Chain (Threonin B30) is removed. Furthermore, a glutamic acid and 16C-fatty acid were added to Lysin B29.

In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise. The similarity of the amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available e.g. on http://www.ebi.ac.uk/Tools/clustalw/ or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215: 403-410. BLAST protein searches are performed with the BLASTP program, score=50, word length=3, to obtain amino acid sequences homologous to the parent polypeptide. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:I54-I62) or Markov random fields. When percentages of sequence identity are referred to in the present application, these percentages are calculated in relation to the full length of the longer sequence, if not specifically indicated otherwise.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment.

Examples of a disease include but are not limited to traumatic diseases, inflammatory diseases, infectious diseases, cutaneous conditions, endocrine diseases, intestinal diseases, neurological disorders, joint diseases, genetic disorders, autoimmune diseases, and various types of cancer.

Typically, but not necessarily, a disease or disorder is associated with specific “symptoms” or “signs” indicating the presence of such disease or injury. The presence of such symptoms or signs may thus, be indicative for a tissue, an organ or an individual suffering from a disease or injury. An alteration of these symptoms or signs may be indicative for the progression of such a disease or injury. A progression of a disease or injury is typically characterised by an increase or decrease of such symptoms or signs which may indicate a “worsening” or “bettering” of the disease or injury. The “worsening” of a disease or injury is characterised by a decreasing ability of a tissue, organ or organism to fulfil its function efficiently, whereas the “bettering” of a disease or injury is typically characterised by an increase in the ability of a tissue, an organ or an individual to fulfil its function efficiently. A tissue, an organ or an individual being at “risk of developing” a disease or injury is in a healthy state but shows potential of a disease or injury emerging. Typically, the risk of developing a disease or injury is associated with early or weak signs or symptoms of such disease. In such case, the onset and/or progression of the disease or injury may still be prevented by treatment.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

Diabetes mellitus (DM) is a serious chronic disease characterized by an elevated blood sugar. Symptoms of a high blood sugar include but are not limited to frequent urination, increased thirst and increased hunger. If left untreated diabetes can cause both acute and long-term complications. Acute complications include but are not limited to diabetic ketoacidosis and non-ketotic hyperosmolar states. Long-term consequences include but are not limited to heart attacks, strokes, peripheral vascular disease, TIA's, renal insufficiency, renal failure, chronic neuropathic pain, foot ulceration, amputations, blindness, retinal damage, cataracts, fractures, cognitive decline, non-alcoholic steatohepatitis, cirrhosis and a variety of cancers. Diabetes occurs because the pancreas is unable to make sufficient insulin to keep glucose levels normal in both the fasting and the fed state and many people with diabetes also have cells that are not responding properly to the insulin that is produced. There are 3 main types of diabetes mellitus:

Type 1 diabetes mellitus results from the pancreas's failure to produce enough insulin. This form was previously referred to as “insulin-dependent diabetes mellitus” (IDDM) or “juvenile diabetes”. Type 1 diabetes mellitus is characterized by loss of the insulin-producing beta cells of the islets of Langerhans in the pancreas, leading to insulin deficiency. This type can be further classified as immune-mediated or idiopathic. The majority of type 1 diabetes is of the immune-mediated nature, in which a T-cell-mediated autoimmune attack leads to the loss of beta cells and thus insulin.

Type 2 diabetes occurs when the pancreas is not able to make enough insulin to overcome resistance to the action of insulin. This form was previously referred to as “non insulin-dependent diabetes mellitus” (NIDDM) or “adult-onset diabetes”. The primary cause is excessive body weight and not enough exercise. Type 2 diabetes mellitus is characterized by insulin resistance, which may be combined with relatively reduced insulin secretion. The defective responsiveness of body tissues to insulin is believed to involve the insulin receptor. However, the specific defects are not known. Type 2 diabetes mellitus is the most common type of diabetes mellitus. In the early stage of type 2, the predominant abnormality is reduced insulin sensitivity. At this stage, high blood sugar can be reversed by a variety of measures and medications that improve insulin sensitivity or reduce the liver's glucose production.

Gestational diabetes is the third main form and occurs when pregnant women without a previous history of diabetes develop a high blood-sugar level. Gestational diabetes mellitus (GDM) resembles type 2 diabetes mellitus in several respects, involving a combination of relatively inadequate insulin secretion and responsiveness. It occurs in about 2-10% of all pregnancies and may improve or disappear after delivery. However, after pregnancy approximately 5-10% of women with gestational diabetes are found to have diabetes mellitus, most commonly type 2. Gestational diabetes is fully treatable, but requires careful medical supervision throughout the pregnancy.

Prediabetes indicates a condition that occurs when a person's blood glucose levels are higher than normal but not high enough for a diagnosis of type 2 diabetes. Many people destined to develop type 2 diabetes mellitus spend many years in a state of prediabetes.

As noted above diabetes is associated with a large variety of serious long-term consequences. Besides others, these include but are not limited to cardiovascular diseases.

The term “cardiovascular disease (CVD)” refers to a group of disorders of the heart and blood vessels and include but are not limited to coronary heart disease, cerebrovascular disease, peripheral arterial disease, rheumatic heart disease, congenital heart disease, and deep vein thrombosis and pulmonary embolism. The term “coronary heart disease” refers to diseases of the blood vessels supplying the heart muscle. The term “cerebrovascular disease” refers to diseases of the blood vessels supplying the brain. The term “peripheral arterial disease” refers to diseases of blood vessels supplying the arms and legs. The term “rheumatic heart disease” refers to damage to the heart muscle and heart valves from rheumatic fever, in particualer rheumatic fever caused by streptococcal bacteria. The term “congenital heart disease” refers to malformations of heart structure existing at birth. The term “deep vein thrombosis and pulmonary embolism” refers to blood clots in the leg veins, which can dislodge and move to the heart and lungs.

Within cardiovascular diseases, “heart attacks” and “strokes” are usually acute events and are mainly caused by a blockage that prevents blood from flowing to the heart or brain. The most common reason is a build-up of fatty deposits on the inner walls of the blood vessels. Strokes can be caused by bleeding from a blood vessel in the brain or by blood clots. Cardiovascular diseases are the leading cause of death globally. “Myocardial infarction (MI)” or “acute myocardial infarction (AMI)”, commonly known as a heart attack, occurs when blood flow stops to a part of the heart causing damage to the heart muscle. The most common symptom is chest pain or discomfort which may travel into the shoulder, arm, back, neck, or jaw. Often it is in the center or left side of the chest and lasts for more than a few minutes. The discomfort may occasionally feel like heartburn. Other symptoms may include shortness of breath, nausea, feeling faint, a cold sweat, or feeling tired. About 30% of people have atypical symptoms, with women more likely than men to present atypically. Among those over 75 years old, about 5% have had an MI with little or no history of symptoms. An MI may cause heart failure, an irregular heartbeat, or cardiac arrest. Most MIs occur due to coronary artery disease. Risk factors include but are not limited to high blood pressure, smoking, diabetes, lack of exercise, obesity, high blood cholesterol, poor diet, and excessive alcohol intake. The mechanism of an MI often involves the rupture of an atherosclerotic plaque, leading to complete blockage of a coronary artery. A number of tests are useful to help with diagnosis, including but not limited to electrocardiograms (ECGs), blood tests, and coronary angiography.

The term “angina pectoris” or “angina” refers to the sensation of chest pain, pressure, or squeezing, often due to ischemia of the heart muscle from obstruction or spasm of the coronary arteries. While angina pectoris can derive from anemia, abnormal heart rhythms and heart failure, its main cause is coronary artery disease, an atherosclerotic process affecting the arteries feeding the heart. Stable angina, also known as effort angina, refers to the classic type of angina related to myocardial ischemia. A typical presentation of stable angina is that of chest discomfort and associated symptoms precipitated by some activity (running, walking, etc.) with minimal or non-existent symptoms at rest or after administration of sublingual nitroglycerin. In contrast, unstable angina (UA) (also “crescendo angina”; this is a form of acute coronary syndrome) is defined as angina pectoris that changes or worsens. UA may occur unpredictably at rest, which may be a serious indicator of an impending heart attack. Stable angina is differentiated from unstable angina (other than symptoms) is the underlying pathophysiology of the atherosclerosis. The pathophysiology of unstable angina is the reduction of coronary flow due to transient platelet aggregation on apparently normal endothelium, coronary artery spasms, or coronary thrombosis. The process starts with atherosclerosis, progresses through inflammation to yield an active unstable plaque, which undergoes thrombosis and results in acute myocardial ischemia, which, if not reversed, results in cell necrosis (infarction). In stable angina, the developing atheroma is protected with a fibrous cap. This cap may rupture in unstable angina, allowing blood clots to precipitate and further decrease the area of the coronary vessel's lumen.

Within cardiovascular diseases, the term “heart failure” is included but may have a descrete meaning. As consequence of long-lasting cardiovascular disease or as a consequence of a massive heart attack, the cardiac muscle may be damaged to a degree that it is too much weakened and cannot pump enough blood to meet the body's needs for blood and oxygen. When heart failure is caused by heart damage that has developed over time, it cannot be cured. Heart failure is one of the most common reasons for hospital admissions among those 65 years and older. Individuals with diabetes as co-morbidity are not only at high risk of developing heart failure but are also at increased risk of dying from it. Fortunately, typical cardiovascular therapies such as angiotensin-converting-enzyme inhibitors, β blockers and mineralocorticoid-receptor antagonists work similarly well in heart failure patients without and with diabetes. However, response to intensive glycaemic control and the various classes of antihyperglycaemic agent therapy used normally to treat Diabetes is substantially less well understood. The need for new glucose-lowering drugs to show cardiovascular safety has led to the unexpected finding of an increase in the risk of admission to hospital for heart failure in patients treated with the dipeptidylpeptidase-4 (DPP4) inhibitor, saxagliptin, compared with placebo. The term “heart failure” includes a bundle of different clinical symptoms and their definitions, such as chronic heart failure, acute heart failure, congestive heart failure, idiopathic heart failure, cardiac dysfunction, cardiomyopathy, and diabetic cardiomyopathy,

The term “cardiac stroke volume” refers to the volume of blood pumpt per stroke.

The term “cardiac efficiency” is defined as the proportion of work done by the heart muscle in relation to the energy used by the heart muscle to perform this work.

In cardiac physiology, the term “stroke work” refers to the work done by the left ventricular heart muscle to eject a volume of blood, i.e. stroke volume, into the aorta. Stroke work can be estimated as the product of cardiac stroke volume and mean aortic pressure during ejection, and is sometimes used to assess ventricular function.

The term “cardiac output” describes in cardiac physiology the volume of blood being pumped by the heart, per unit time. Because cardiac output is related to the quantity of blood delivered to various parts of the body, it is a key indicator of how efficiently the heart can meet the demands of the body. Along with the term “stroke volume”, cardiac output is a central parameter of interest in hæmodynamics—the study of the flow of blood under external forces.

Cardiac output and cardiac stroke volume may be measured via “echocardiography”, i.e. via a sonogram of the heart. Echocardiography uses standard two-dimensional, three-dimensional, alone or combined with and Doppler ultrasound, to create images of the heart, and is routinely used in the diagnosis, management, and follow-up of patients with any suspected or known heart diseases. It is one of the most widely used diagnostic tests in cardiology^{5, 6}. Besides providing information concerning the size and shape of the heart (e.g. internal chamber size quantification), pumping capacity, and the location and extent of any tissue damage, an echocardiogram also allows for the evaluation of heart functions such as the cardiac output, ejection fraction, and diastolic function (how well the heart relaxes). Echocardiography allows for the detection of cardiomyopathies, such as hypertrophic cardiomyopathy, dilated cardiomyopathy, and many others. The use of Stress Echocardiography may also help determine whether any chest pain or associated symptoms are related to heart disease. The biggest advantage to echocardiography is that it is noninvasive (doesn't involve breaking the skin or entering body cavities) and has no known risks or side effects. In particular, Doppler echocardiography using pulsed or continuous wave Doppler ultrasound allows for an accurate assessment of the blood flowing through the heart. Color Doppler as well as spectral Doppler is commonly used to visualize any abnormal communications between the left and right side of the heart, any leaking of blood through the valves (valvular regurgitation), and to estimate how well the valves open (or do not open in the case of valvular stenosis). The Doppler technique can also be used for tissue motion and velocity measurement, by Tissue Doppler echocardiography.

Cellular respiration is the set of metabolic reactions and processes that take place in the cells of organisms to convert biochemical energy from nutrients into adenosine triphosphate (ATP). Nutrients that are commonly used by animal and plant cells in respiration include sugar, amino acids and fatty acids, and the most common oxidizing agent (electron acceptor) is molecular oxygen (O₂). The heart has a very high energy demand and continually generate ATP at a high rate to sustain contractile function, basal metabolic processes, and ionic homeostasis. In the normal adult heart, almost all (˜95%) of ATP production is derived from mitochondrial oxidative phosphorylation which includes fatty acid β-oxidation and glucose oxidation.

In the heart, the term “glucose oxidation” refers to the energy generation from glucose. Glucose oxidation is a cellular biochemical process that uses oxygen to oxidize glucose to carbon dioxide and water; the chemical energy released from oxidation is used to generate ATP. Glucose oxidation comprises four stages. During Glycolysis, one glucose molecule is broken down into two pyruvates and energy is stored in the form of two molecules of NADH and ATP, respectively. In the second stage, the pyruvate is processed by pyruvate dehydrogenase to generate acetyl-CoA, carbon dioxide and NADH. The third stage is the Krebs cycle where acetyl-CoA is oxidized into carbon dioxide and NADH, FADH2 and ATP or GTP. The last stage is oxidative phosphorylation, where all the NADH and FADH2 molecules are oxidized to ATP. Glucose uptake and/or glucose oxidation measurements are established using positron emission tomography of radioactive glucose derivatives as a nuclear medicine. Magnetic resonance spectroscopy with hyperpolarized radioactive pyruvate is a technique capable of detecting changes in glucose oxidation. Herein, based on dynamic nuclear polarization and in combination with metabolic tracers of 13C-labelled pyruvate, this measurement protocol has been used and validated to assess changes in myocardial metabolism in the normal and diseased heart in vivo in different models of diabetes, ischaemic heart disease, cardiac hypertrophy and heart failure⁷ and safe application of this technology in humans has been demonstrated⁸.

Also simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation.

The term “fatty acid oxidation” or “beta-oxidation” refers to the energy generation from fatty acids. Beta-oxidation results in the formation of acetyl-CoA, FADH2 and NADH, which are finally oxidized in the Krebs cycle or the oxidative phosphorylation reaction. The process consists of several cycles, with one cycle having 4 enzymatic steps. The process continues until all of the carbons in the fatty acid are turned into acetyl CoA. For example palmitic acid consist of a 16 carbon acyl chain, thus 8 cycles are needed to fully oxidize this fatty acid with its particular chain length. Under normal conditions, the majority of the acetyl-CoA that enters the Krebs cycle is generated by beta-oxidation of FA, while about a third is derived from oxidation of pyruvate. In type 2 diabetes a shift in oxidation towards FA, at the cost of glucose occurred. At present, this metabolic disturbances due to lipid overload are thought to be one underlying cause of cardiac dysfunction in type 2 diabetes.

As used herein, “treat”, “treating” or “treatment” of a disease or injury means accomplishing one or more of the following: (a) reducing the severity of the disease or injury; (b) limiting or preventing development of symptoms characteristic of the disease or injury being treated; (c) inhibiting worsening of symptoms characteristic of the disease or injury being treated; (d) limiting or preventing recurrence of the disease or injury in an individual who has previously had the disease or injury; and (e) limiting or preventing recurrence of symptoms in individuals who were previously symptomatic for the disease or injury.

As used herein, “prevent”, “preventing”, “prevention”, or “prophylaxis” of a disease or injury means preventing that such disease or injury occurs in patient.

The term “delay” or “delaying” a disease refers to a descrese in the progression of disease or disorder, i.e. delaying a disease refers to prolonging the time frame in which the symptoms or signs or cause of the disease worsen.

The terms “pharmaceutical”, “pharmaceutical composition”, “medicament” and “drug” are used interchangeably herein referring to a substance and/or a combination of substances being used for the identification, prevention or treatment of a disease or injury.

As used herein, “administering” includes in vivo administration, as well as administration directly to tissue ex vivo, such as vein grafts.

An “effective amount” is an amount of an agent sufficient to achieve the intended purpose. The effective amount of a given agent may vary due to factors such as the nature of the agent, the route of administration, the size and species of the subject to receive the agent, and the purpose of the administration. The effective amount in each individual case may be determined empirically by a skilled artisan according to established methods in the art.

The term “therapeutically effective amount” is an amount of a therapeutic agent sufficient to achieve the intended preventive or therapeutic effect, i.e. the amount of said therapeutic agent which is considered to achieve a bettering of the signs of symtomes of the disorder or disease to be treated, or to prevent the onset of the signs of symtomes of a disorder or disease to be prevented. The effective amount of a given therapeutic agent may vary due to factors such as the nature of the agent, the route of administration, the size and species of the animal to receive the therapeutic agent, and the purpose of the administration. The therapeutically effective amount in each individual case may be determined empirically by a skilled artisan according to established methods in the art.

“Pharmaceutically acceptable” means approved by a regulatory agency of the Federal or a state government or listed in the U.S. Pharmacopeia or other generally recognized pharmacopeia for use in animals, and more particularly in humans.

The term “active ingredient” refers to the substance in a pharmaceutical composition or formulation that is biologically active, i.e. that provides pharmaceutical value. A pharmaceutical composition may comprise one or more active ingredients which may act in conjunction with or independently of each other. The active ingredient can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylamine, 2-ethylamino ethanol, histidine, procaine, and the like.

The terms “preparation” and “composition” are intended to include the formulation of the active compound with encapsulating material as a carrier providing a capsule in which the active component with or without other carriers, is surrounded by a carrier, which is thus in association with it.

The term “carrier”, as used herein, refers to a pharmacologically inactive substance such as but not limited to a diluent, excipient, or vehicle with which the therapeutically active ingredient is administered. Such pharmaceutical carriers can be liquid or solid. Liquid carrier include but are not limited to sterile liquids, such as saline solutions in water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The term “adjuvant” refers to agents that augment, stimulate, activate, potentiate, or modulate the therapeutic effect of the active ingredient. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g polyarginine or polylysine).

As used herein, a “patient” means any mammal, reptile or bird that may benefit from the present invention. Preferably, a patient is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. It is particularly preferred that the “patient” is a human being.

### Embodiments

The diabetic heart is insulin resistant and has increased fatty acid oxidation and decreased glucose oxidation. These changes in energy metabolism decrease cardiac efficiency and contribute to the decreased function of diabetic hearts. The ability of insulin to stimulating glucose oxidation and inhibit fatty acid oxidation is beneficial, especially in the diabetic heart. There is evidence that improving cardiac output, such as by stimulating glucose oxidation, improves cardiac function.

In a first aspect, the present invention provides an insulin analogue for use in increasing cardiac output or cardiac stroke volume in a patient, in particular in a patient at risk of developing or suffering from diabetes. Thus, the present invention provides an insulin analogue for use in increasing cardiac output or cardiac stroke volume in a patient at risk of developing or suffering from diabetes. In particular embodiments, said patient is at risk of developing or suffering from type I diabetes or type II diabetes. In particular embodiments, the patient exhibits a decreased cardiac output or a decreased stroke volume, and/or exhibits increased fatty acid oxidation and/or reduced glucose oxidation in the heart. In particular embodiments, the patient suffers from diabetes and has already experienced a cardiovascular disease, in particular a heart failure.

In embodiments, the insulin analogue increases cardiac output by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the the insulin analogue increases the cardiac output by 20-30%, in particular by 22-26%. In particular embodiments, the insulin analogue increases the cardiac output by 20%, 22%, 24% or 26%. In particular embodiments, the insulin analogue increases the cardiac output in chronic treatment.

In embodiments, the insulin analogue increases the cardiac stroke volume by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the the insulin analogue increases the cardiac stroke volume by 20-30%, in particular by 22-28%. In particular embodiments, the insulin analogue increases the cardiac stroke volume by 22%, 24%, 26% or 28%.

In particular embodiments, the insulin analogue increases the glucose oxidation and/or decreases the fatty acid oxidation in the heart.

Thus, in particular embodiments, the insulin analogue is for acute and/or chronic use in increasing cardiac efficiency or cardiac stroke volume, in particular in a patient at risk of developing or suffering from diabetes. The skilled person will appreciate that the precise increase in the cardiac output and/or the cardiac stroke volume is dependent on the initial cardiac function of the particular patient and may thus vary. Further, the skilled person will appreciate that the precise increase in the cardiac efficacy and/or the cardiac stroke volume is confined by the sensitivity of the different clinical methods used to measure cardiac output and stroke volume.

In particular embodiments, the cardiac output and/or cardiac stroke volume is assessed via imaging technologies, in particular via cardiac echography. In particular embodiments, Doppler Ultrasound may be used in combination with echocardiography to give a more complete picture of blood flow to the heart. Alternatively or in combination, cardiac function can be assessed and/or confirmed by cardiac magnetic resonance imaging and/or by determining the level of biomarker such as e.g. brain natriuretic peptide (BNP) and precursors, analogs and fragments of BNP.

In particular embodiments, glucose uptake and/or glucose oxidation measurements may be performed using magnetic resonance spectroscopy comprising the use of hyperpolarized radioactive pyruvate. Also, simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation. In particular embodiments, the the insulin analogue is derived from human insulin.

In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural insulin at position A21 to Gly, and wherein two Arg are added to the C-terminus of the B-chain (insulin glargine, Gly (A21), Arg (B31), Arg (B32) human insulin). In other embodiments, the insulin analogue is Glargine-M1 (Gly (A21) human insulin) wherein the two additional Arg residues are removed but A21 is still Gly.

In particular embodiments the patient is a mammal, reptile or bird that may benefit from the present invention. In particular, the patient is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. In particular, the patient is a human being.

In a second aspect, the present invention provides an insulin analogue for use in preventing, delaying and/or treating cardiovascular diseases in patients at risk of developing or suffering from diabetes. In particular embodiments, said patient is at risk of developing or suffering from type I diabetes or type II diabetes. In particular embodiments, the patient exhibits a decreased cardiac output or a decreased stroke volume, and/or exhibits increased fatty acid oxidation and/or reduced glucose oxidation in the heart. In particular embodiments, the patient suffers from diabetes and has already experienced a cardiovascular disease, in particular a heart failure.

In particular embodiments, the cardiovascular disease is selected from the group consisting of cardiovascular disorders accompanied by decreased cardiac function. In particular embodiments, the cardiovascular disease is selected from the group consisting of heart failure, coronary heart disease, cerebrovascular disease, peripheral arterial disease, rheumatic heart disease, congenital heart disease, and deep vein thrombosis and pulmonary embolism.

In particular embodiments, the insulin analogue increases the glucose oxidation and/or decreases the fatty acid oxidation in the heart, thereby preventing and/or treating cardiovascular diseases.

In particular embodiments, the insulin analogue increases cardiac output and/or cardiac stroke volume of the heart, thereby preventing the progression of and/or treating cardiovascular diseases, as well as delaying cardiovascular outcomes.

In embodiments, the insulin analogue increases cardiac output by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the insulin analogue increases the cardiac output by 20-30%, in particular by 22-26%. In particular embodiments, the insulin analogue increases the cardiac output by 20%, 22%, 24% or 26%. In particular embodiments, the insulin analogue increases the cardiac output in chronic treatment.

In embodiments, the insulin analogue increases the cardiac stroke volume by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the insulin analogue increases the cardiac stroke volume by 20-30%, in particular by 22-28%. In particular embodiments, the insulin analogue increases the cardiac stroke volume by 22%, 24%, 26% or 28%.

The skilled person will appreciate that the precise increase in the cardiac output and/or the cardiac stroke volume is dependent on the initial cardiac function of the particular patient and may thus vary. Further, the skilled person will appreciate that the precise increase in the cardiac output and/or the cardiac stroke volume is confined by the sensitivity of the different clinical methods used to measure cardiac output and stroke volume.

In particular embodiments, the cardiac output and/or cardiac stroke volume is assessed via imaging technologies, in particular via cardiac echography. In particular embodiments, Doppler Ultrasound may be used in combination with echocardiography to give a more complete picture of blood flow to the heart. Alternatively or in combination, cardiac function can be assessed and/or confirmed by cardiac magnetic resonance imaging and/or by determining the level of biomarker such as e.g. brain natriuretic peptide (BNP) and precursors, analogs and fragments of BNP.

In particular embodiments, glucose uptake and/or glucose oxidation measurements may be performed using magnetic resonance spectroscopy comprising the use of hyperpolarized radioactive pyruvate. Also, simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation. In particular embodiments, the the insulin analogue is derived from human insulin.

Thus, in particular embodiments, the insulin analogue, is for use in preventing and/or treating cardiovascular diseases, and/or for for delaying cardiovascular outcomes, wherein the cardiovascular disease is prevented, treated or delayed by increasing the cardiac efficacy and/or the cardiac stroke volume of the heart, in particular by increasing the cardiac output by 24% and/or the cardiac stroke volume by 26% in a patient at risk of developing or suffering from diabetes.

In particular embodiments, the insulin analogue is derived from human insulin. In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural at position A21 to Gly, and wherein two Arg are added to the C-terminus of the B-chain. In particular embodiments, the insulin analogue is Glargine or Glargine-M1.

In particular embodiments, the patient is a mammal, reptile or bird that may benefit from the present invention. In particular, the patient is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. In particular, the patient is a human being.

In a third aspect, the present invention relates to a method of preventing, delaying, and/or treating cardiovascular diseases in patients at risk of developing or suffering from diabetes comprising administering a therapeutically effective amount of an insulin analogue to the patient. In particular embodiments, the diabetes is type I or type II diabetes. In particular embodiments, the patient exhibits a decreased cardiac output or a decreased stroke volume, and/or exhibits increased fatty acid oxidation and/or reduced glucose oxidation in the heart. In particular embodiments, the patient suffers from diabetes and has already experienced a cardiovascular disease, in particular heart failure.

In particular embodiments, the cardiovascular disease is selected from the group consisting of cardiovascular disorders accompanied by decreased cardiac function. In particular embodiments, the cardiovascular disease is selected from the group consisting of heart failure, coronary heart disease, cerebrovascular disease, peripheral arterial disease, rheumatic heart disease, congenital heart disease, and deep vein thrombosis and pulmonary embolism.

In particular embodiments, the insulin analogue increases cardiac output and/or cardiac stroke volume of the heart, thereby preventing the progression of and/or treating cardiovascular diseases, as well as delaying cardiovascular outcomes.

In embodiments, the insulin analogue increases cardiac output by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the the insulin analogue increases the cardiac output by 20-30%, in particular by 22-26%. In particular embodiments, the insulin analogue increases the cardiac output by 20%, 22%, 24% or 26%. In particular embodiments, the insulin analogue increases the cardiac output in chronic treatment.

In embodiments, the insulin analogue increases the cardiac stroke volume by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the the insulin analogue increases the cardiac stroke volume by 20-30%, in particular by 22-28%. In particular embodiments, the insulin analogue increases the cardiac stroke volume by 22%, 24%, 26% or 28%.

The skilled person will appreciate that the precise increase in the cardiac output and/or the cardiac stroke volume is dependent on the initial cardiac function of the particular patient and may thus vary. Further, the skilled person will appreciate that the precise increase in the cardiac output and/or the cardiac stroke volume is confined by the sensitivity of the different clinical methods used to measure cardiac output and stroke volume.

In particular embodiments, the cardiac output and/or cardiac stroke volume is assessed via imaging technologies, in particular via cardiac echography. In particular embodiments, Doppler Ultrasound may be used in combination with echocardiography to give a more complete picture of blood flow to the heart. Alternatively or in combination, cardiac function can be assessed and/or confirmed by cardiac magnetic resonance imaging and/or by determining the level of biomarker such as e.g. brain natriuretic peptide (BNP) and precursors, analogs and fragments of BNP.

In particular embodiments, glucose uptake and/or glucose oxidation measurements may be performed using magnetic resonance spectroscopy comprising the use of hyperpolarized radioactive pyruvate. Also, simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation. In particular embodiments, the the insulin analogue is derived from human insulin.

In particular embodiments, the insulin analogue increases the glucose oxidation and/or decreases the fatty acid oxidation in the heart. Thus, in particular embodiments, the cardiovascular diseases is prevented and/or treated in patients at risk of developing or suffering from diabetes by administering a therapeutically effective amount of an insulin analogue, wherein said insulin analogue increases the glucose oxidation and/or decreases the fatty acid oxidation.

Accordingly, in particular embodiments, the method of treating preventing, delaying, and/or treating cardiovascular diseases in patients at risk of developing or suffering from diabetes comprises the step of determining the cardiac output, cardiac stroke volume, and/or the glucose uptake and/or the glucose oxidation prior to the administration of the insulin analogue.

In particular embodiments, the insulin analogue is derived from human insulin. In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural at position A21 to Gly, and wherein two Arg are added to the C-terminus of the B-chain. In particular embodiments, the insulin analogue is Glargine or Glargine-M1.

In particular embodiments the patient is a mammal, reptile or bird that may benefit from the present invention. In particular, the patient is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. In particular, the patient is a human being.

In a fourth aspect, the present invention relates to a pharmaceutical composition for use in increasing cardiac output or cardiac stroke volume or for use in preventing and/or treating cardiovascular diseases comprising an insulin analogue and at least one pharmaceutical acceptable carrier, adjuvant and/or excipient.

In particular embodiments, the insulin analogue is derived from human insulin. In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural at position A21 to Gly, and wherein two Arg are added to the C-terminus of the B-chain. In particular embodiments, the insulin analogue is Glargine or Glargine-M1.

In particular embodiments, the cardiovascular disease is selected from the group consisting of cardiovascular disorders accompanied by decreased cardiac function. In particular embodiments, the cardiovascular disease is selected from the group consisting of heart failure, coronary heart disease, cerebrovascular disease, peripheral arterial disease, rheumatic heart disease, congenital heart disease, and deep vein thrombosis and pulmonary embolism.

In particular embodiments, the pharmaceutical composition increases cardiac output and/or cardiac stroke volume of the heart, thereby preventing the progression of and/or treating cardiovascular diseases, as well as delaying cardiovascular outcomes.

In embodiments, the pharmaceutical composition increases cardiac output by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the pharmaceutical composition increases the cardiac output by 20-30%, in particular by 22-26%. In particular embodiments, the pharmaceutical composition increases the cardiac output by 20%, 22%, 24% or 26%. In particular embodiments, the pharmaceutical composition increases the cardiac output in chronic treatment.

In embodiments, the pharmaceutical composition increases the cardiac stroke volume by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the pharmaceutical composition increases the cardiac stroke volume by 20-30%, in particular by 22-28%. In particular embodiments, pharmaceutical composition increases the cardiac stroke volume by 22%, 24%, 26% or 28%.

The skilled person will appreciate that the precise increase in the cardiac output and/or the cardiac stroke volume is dependent on the initial cardiac function of the particular patient and may thus vary. Further, the skilled person will appreciate that the precise increase in the cardiac output and/or the cardiac stroke volume is confined by the sensitivity of the different clinical methods used to measure cardiac output and stroke volume.

Thus, in particular embodiments, the pharmaceutical composition is for use in increasing cardiac output and/or cardiac stroke volume, wherein the cardiac output is increased by 20-30% and/or the cardiac stroke volume is increased by 20-30%.

In particular embodiments, the cardiac output and/or cardiac stroke volume is assessed via imaging technologies, in particular via cardiac echography. In particular embodiments, Doppler Ultrasound may be used in combination with echocardiography to give a more complete picture of blood flow to the heart. Alternatively or in combination, cardiac function can be assessed and/or confirmed by cardiac magnetic resonance imaging and/or by determining the level of biomarker such as e.g. brain natriuretic peptide (BNP) and precursors, analogs and fragments of BNP.

In particular embodiments, glucose uptake and/or glucose oxidation measurements may be performed using magnetic resonance spectroscopy comprising the use of hyperpolarized radioactive pyruvate. Also, simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation. In particular embodiments, the the insulin analogue is derived from human insulin.

In particular embodiments, the pharmaceutical composition is for use in a patient at risk of developing or suffering from diabetes. In particular embodiments, said patient is at risk of developing or suffering from type I diabetes or type II diabetes. In particular embodiments, the pharmaceutical composition is for use in a patient who exhibit a decreased cardiac output or a decreased stroke volume, and/or who exhibits increased fatty acid oxidation and/or reduced glucose oxidation in the heart. In particular embodiments, the pharmaceutical composition is for use in a patient who suffers from diabetes and who has already experienced a cardiovascular disease, in particular heart failure.

In particular embodiments, the patient is a mammal, reptile or bird that may benefit from the present invention. In particular, the patient is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. In particular, the patient is a human being.

In a fifth aspect, the present invention provides a method of identifying a patient or a group of patients who may benefit from treatment with an insulin analogue, wherein said patient is at risk of developing or suffering from diabetes, comprising the step of determining whether said patient is at risk of developing or suffering from decreased cardiac output or decreased cardiac stroke volume or from a cardiovascular disease.

In particular embodiments, the said patient or group of patients is at risk of developing or suffering from type I diabetes or type II diabetes. In particular embodiments, the patient or group of patients exhibits increased fatty acid oxidation and/or reduced glucose oxidation in the heart. In particular embodiments, the patient or the group of patients suffers from diabetes and has already experienced a cardiovascular disease, in particular heart failure.

In particular embodiments, the cardiovascular disease is selected from the group consisting of cardiovascular disorders accompanied by decreased cardiac function. In particular embodiments, the cardiovascular disease is selected from the group consisting of heart failure, coronary heart disease, cerebrovascular disease, peripheral arterial disease, rheumatic heart disease, congenital heart disease, and deep vein thrombosis and pulmonary embolism.

In embodiments, the patient exhibits a cardiac output decreased by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the patient exhibits a cardiac output decreased by 20-30%, in particular by 22-26%. In particular embodiments, the patient exhibits a cardiac output decreased by 20%, 22%, 24% or 26%.

In embodiments, the patient exhibits a cardiac stroke volume decreased by 15-40%, i.e. 15%, 16%, 17%, 18%, 19%, 20%, 21%, 22,%, 23%, 24%, 25%, 26%, 27%, 28%, 29%, 30%, 31%, 32%, 33%, 34%, 35%, 36%, 37%, 38%, 39%, or 40%. In particular embodiments, the patient exhibits a cardiac stroke volume decreased by 20-30%, in particular by 22-28%. In particular embodiments, the patient exhibits a cardiac stroke volume decreased by 22%, 24%, 26% or 28%.

In particular embodiments, the patient exhibits a decreased cardiac output, in particular a cardiac output which is decreased by 20-30%. In particular embodiments, the patient exhibits a decreased cardiac stroke volume, in particular a decreased stroke volume, which is decreased by 20-30%.

In particular embodiments, the cardiac output and/or cardiac stroke volume is assessed via imaging technologies, in particular via cardiac echography. In particular embodiments, Doppler Ultrasound may be used in combination with echocardiography to give a more complete picture of blood flow to the heart. Alternatively or in combination, cardiac function can be assessed and/or confirmed by cardiac magnetic resonance imaging and/or by determining the level of biomarker such as e.g. brain natriuretic peptide (BNP) and precursors, analogs and fragments of BNP.

In particular embodiments, the insulin analogue is derived from human insulin. In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural at position A21 to Gly, and wherein two Arg are added to the C-terminus of the B-chain. In particular embodiments, the insulin analogue is Glargine or Glargine-M1.

Accordingly, the method of identifying a patient or a group of patients who will benefit from treatment with an insulin analogue, comprises the step of determining the cardiac output and/or the cardiac stroke volume, in particular using imaging technologies, in particular via cardiac echography.

In a sixth aspect, the present invention relates to a method of identifying a patient or a group of patients who will benefit from treatment with an insulin analogue, wherein said patient or said group of patients is at risk of developing or suffering from diabetes and exhibits increased fatty acid oxidation and/or reduced glucose oxidation in the heart.

In particular embodiments, the insulin analogue is derived from human insulin. In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural at position A21 to Gly, and wherein two Arg are added to the C-terminus of the B-chain. In particular embodiments, the insulin analogue is Glargine or Glargine-M1.

In particular embodiments, said patient or said group of patients is at risk of developing or suffering from type I diabetes or type II diabetes. In particular embodiments, the patient or group of patients exhibits a decreased cardiac output or a decreased stroke volume. In particular embodiments, the patient or said group of patients suffers from diabetes and has already experienced a cardiovascular disease, in particular heart failure.

In particular embodiments, the cardiovascular disease is selected from the group consisting of cardiovascular disorders accompanied by decreased cardiac function. In particular embodiments, the cardiovascular disease is selected from the group consisting of heart failure, coronary heart disease, cerebrovascular disease, peripheral arterial disease, rheumatic heart disease, congenital heart disease, and deep vein thrombosis and pulmonary embolism.

In particular embodiments, glucose uptake and/or glucose oxidation measurements may be performed using magnetic resonance spectroscopy comprising the use of hyperpolarized radioactive pyruvate. Also, simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation. In particular embodiments, the the insulin analogue is derived from human insulin.

Accordingly, the method of identifying a patient or a group of patients who will benefit from treatment with an insulin analogue, comprises the step of measuring the level of glucose oxidation in the patient, in particular using magnetic resonance spectroscopy, in particular comprising the use of hyperpolarized radioactive pyruvate.

In a seventh aspect, the present invention relates to an insulin analogue for use in preventing, delaying and/or treating a cardiovascular disease in a patient at risk of developing or suffering from diabetes, wherein said patient exhibits increased fatty acid oxidation and/or reduced glucose oxidation.

In particular embodiments, the insulin analogue is derived from human insulin. In particular embodiments, the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension. In particular embodiments, the insulin analogue is modified in comparison to natural at position A21 to Gly, and wherein two Arg are added to the C-terminus of the B-chain. In particular embodiments, the insulin analogue is Glargine or Glargine-M1.

In particular embodiments, the said patient is at risk of developing or suffering from type I diabetes or type II diabetes. In particular embodiments, the patient exhibits a decreased cardiac output or a decreased stroke volume. In particular embodiments, the patient suffers from diabetes and has already experienced a cardiovascular disease, in particular heart failure.

In particular embodiments, the cardiovascular disease is selected from the group consisting of cardiovascular disorders accompanied by decreased cardiac function. In particular embodiments, the cardiovascular disease is selected from the group consisting of heart failure, coronary heart disease, cerebrovascular disease, peripheral arterial disease, rheumatic heart disease, congenital heart disease, and deep vein thrombosis and pulmonary embolism.

In particular embodiments, glucose uptake and/or glucose oxidation measurements may be performed using magnetic resonance spectroscopy comprising the use of hyperpolarized radioactive pyruvate. Also, simultaneous measurements of biochemical plasma biomarkers, i.e. levels of brain natriuretic peptide (BNP) and precursors, analogs and fragments of BNP, in combination with non-invasive imaging technologies are suitable to measure glucose oxidation. In particular embodiments, the the insulin analogue is derived from human insulin.

In particular, the present invention relates to the following items:
1. Insulin analogue for use in increasing cardiac output or cardiac stroke volume in a patient at risk of developing or suffering from diabetes.
2. Insulin analogue for use in increasing cardiac output or cardiac stroke volume according to item 1, wherein the cardiac output is increased by 20-30% and/or the cardiac stroke volume is increased by 20-30%.
3. Insulin analogue for use in preventing, delaying and/or treating cardiovascular diseases in patients at risk of developing or suffering from diabetes.
4. Insulin analogue for use in preventing, delaying and/or treating cardiovascular diseases according to item 3, wherein the cardiovascular disease is selected from the group consisting cardiovascular disorders accompanied by decreased cardiac function.
5. Insulin analogue for use in increasing cardiac output or cardiac stroke volume or for use in preventing, delaying and/or treating cardiovascular diseases according to any of items 1 tor 4, wherein the diabetes is type I or type II diabetes.
6. Insulin analogue for use in increasing cardiac output or cardiac stroke volume or for use in preventing, delaying and/or treating cardiovascular diseases according to any of items 1 tor 5, wherein the insulin analogue increases the glucose oxidation and/or decreases the fatty acid oxidation in the heart.
7. Insulin analogue for use in increasing cardiac output or cardiac stroke volume or for use in preventing, delaying and/or treating cardiovascular diseases according to any of items 1 tor 6, wherein the insulin analogue is derived from human insulin.
8. Insulin analogue for use in increasing cardiac output or cardiac stroke volume or for use in preventing, delaying and/or treating cardiovascular diseases according to any of items 1 tor 7, wherein the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension.
9. Insulin analogue for use in increasing cardiac output or cardiac stroke volume or for use in preventing, delaying and/or treating cardiovascular diseases according to any of items 1 tor 8, wherein the insulin analogue is modified in comparison to natural at position A21 to Gly, and wherein two Arg are added to the C-terminus of the B-chain.
10. Insulin analogue for use in increasing cardiac output or cardiac stroke volume or for use in preventing, delaying and/or treating cardiovascular diseases according to any of items 1 tor 9, wherein the insulin analogue is Glargine or Glargine-M1.
11. Method of preventing, delaying and/or treating cardiovascular diseases in patients at risk of developing or suffering from diabetes comprising administering a therapeutically effective amount of an insulin analogue.
12. Pharmaceutical composition for use in increasing cardiac output or cardiac stroke volume or for use in preventing, delaying and/or treating cardiovascular diseases comprising an insulin analogue and at least one pharmaceutical acceptable carrier, adjuvant and/or excipient.
13. The pharmaceutical composition for use in increasing cardiac output or cardiac stroke volume or for use in preventing, delaying and/or treating cardiovascular diseases according to item 12, wherein the insulin analogue is derived from human insulin.
14. The pharmaceutical composition for use in increasing cardiac output or cardiac stroke volume or for use in preventing, delaying and/or treating cardiovascular diseases according to item 12 or 13, wherein the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension.
15. The pharmaceutical composition for use in increasing cardiac output or cardiac stroke volume or for use in preventing, delaying and/or treating cardiovascular diseases according to any of items 12 to 14, wherein the insulin analogue is modified in comparison to natural insulin at position A21 to Gly, and wherein two Arg are added to the C-terminus of the B-chain.
16. The pharmaceutical composition for use in increasing cardiac output or cardiac stroke volume or for use in preventing and/or treating cardiovascular diseases according to any of items 12 to 15, wherein the insulin analogue is Glargine or Glargine-M1.
17. A method of identifying a patient or a group of patients who may benefit from treatment with an insulin analogue, wherein said patient or said group of patients is at risk of developing or suffering from diabetes, comprising the step of determining whether said patient is at risk of developing or suffering from decreased cardiac output or cardiac stroke volume or cardiovascular disease.
18. The method of item 17, wherein the diabetes is type I or type II diabetes.
19. The method of item 17, wherein the cardiovascular disease is selected from the group consisting of cardiovascular disorders accompanied by decreased cardiac function.
20. The method of any of items 17 to 19, wherein the glucose oxidation is increased and/or the fatty acid oxidation in the heart is decreased.
21. The method of any of items 17 to 20, wherein the cardiac output is decreased by 20-30%.
22. The method of any of items 17 to 21, wherein the insulin analogue is derived from human insulin.
23. The method of any of items 17 to 22, wherein the insulin analogue is modified in comparison to natural insulin at position B28 to one of Asp, Lys, or Ile, wherein Lys at position B29 is modified to Pro, wherein Asn at position A21 is modified to Ala, Gln, Glu, Gly, His, Ile, Leu, Met, Ser, Thr, Trp, Tyr or Val, in particular to Gly, Ala, Ser, or Thr, in particular to Gly, wherein Asn at position B3 may be modified to Lys or Asp, wherein PheBI is deleted, and/or wherein the A-chain and/or the B-chain have a C-terminal and/or N-terminal extension.
24. The method of any of items 17 to 23, wherein the insulin analogue is modified in comparison to natural insulin at position A21 to Gly, and wherein two Arg are added to the C-terminus of the B-chain.
25. The method of any of items 17 to 24, wherein the insulin analogue is Glargine or Glargine-M1.
26. An insulin analogue for use in preventing, delaying and/or treating a cardiovascular disease in a patient at risk of developing or suffering from diabetes, wherein said patient exhibits increased fatty acid oxidation and/or reduced glucose oxidation.
27. A method of identifying a patient or selecting a group of patients who will benefit from treatment with an insulin analogue, wherein said patient or said group of patients is at risk of developing or suffering from diabetes and exhibits increased fatty acid oxidation and/or reduced glucose oxidation in the heart.

The invention is described in more detail in the examples and figures that are not to be understood as limiting the scope of present invention.

### EXAMPLES

The long-term effects of insulin treatment on cardiac function was assessed in two different settings. In acute treatments, measurements were performed in isolated working hearts from either wildtype mice (C57Bl) or mice with a diabetes type-2 phenotype (db/db mice). In a second chronic setting, db/db animals were treated for 4 weeks with daily administration of different high doses of insulins to achieve similar effects on systemic hypergycaemia. At the end of the chronic study, effects on cardiac function was assessed by echocardiography.

### Example 1: Acute Treatment with Lantus Stimulates Cardiac Glucose Oxidation

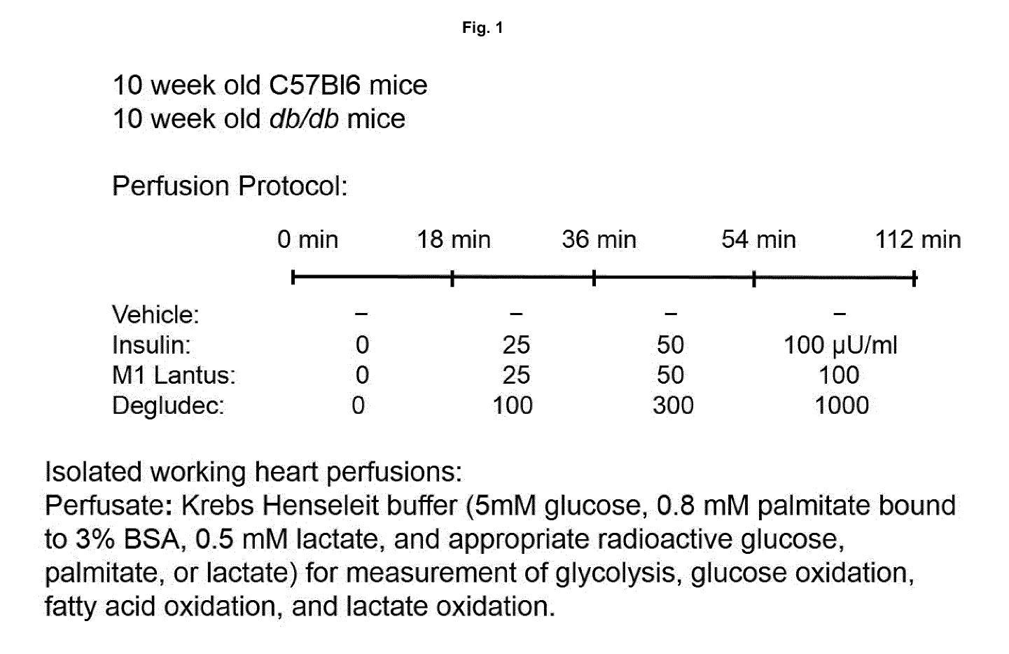

The effect of three different types of insulin, human insulin, insulin degludec, and the M1 metabolite of insulin glargine, on cardiac metabolism was determined in adult, 10 weeks old mice using either healthy C57Bl6 wildtype mice or Type-2 diabetes db/db mice. Mice were fed a standard chow diet and kept in 12 hr light: 12 hr dark cycle. To test their acute effect energy metabolism, hearts were removed from fully anesthetized mice, and perfused as isolated working hearts with Krebs Henseleit buffer (118.5 mM NaCl, 1.2 mM MgSO₄, 25 mM NaHCO₃, 4.7 mM KCl, 1.2 mM KH₂PO₄, 2.5 mM CaCl₂) supplemented with 0.8 mM palmitate bound to 3% fatty acid-free bovine serum albumin (BSA), 5 mM glucose, and 0.5 mM lactate. Hearts underwent aerobic perfusion for 72 min with vehicle or increasing amounts of insulin (0, 25, 50, 100 μU/ml), insulin Degludec (0, 100, 300, 1000 μU/ml), or the M1 metabolite of insulin Glargine (0, 25, 50, 100 μU/ml). Each concentration escalation step lasted 18 minutes. At the end of the heart perfusion hearts were immediately snap frozen in liquid N₂ and stored at −80° C. See FIG. 1

In order to measure glucose oxidation, glycolysis, palmitate oxidation, and lactate oxidation [U-¹⁴C] glucose, [5-³H] glucose, [9, 10-³H] palmitate, and [U-¹⁴C] lactate, respectively, were added to the perfusate as described above in Example 1. Glycolysis and palmitate oxidation rates were determined based on the rates of ³H₂O production.

To determine glucose oxidation and lactate oxidation rates ¹⁴CO₂ production was assessed.

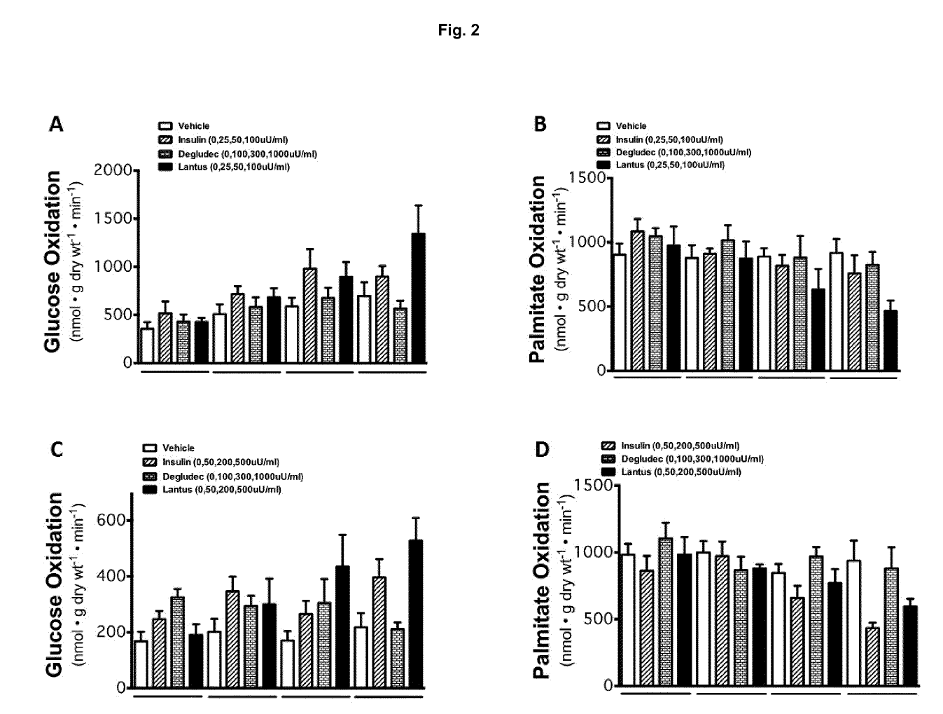

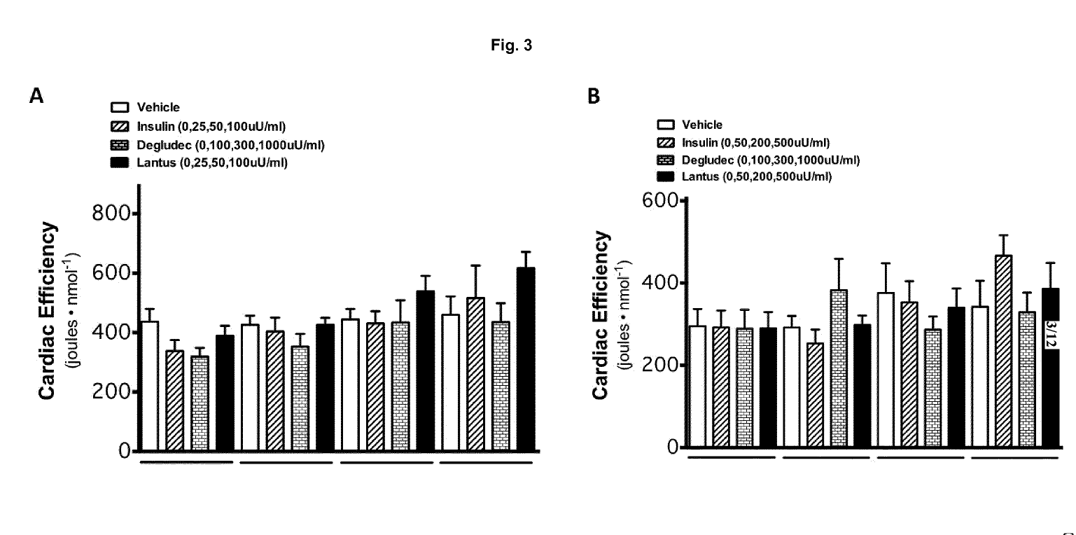

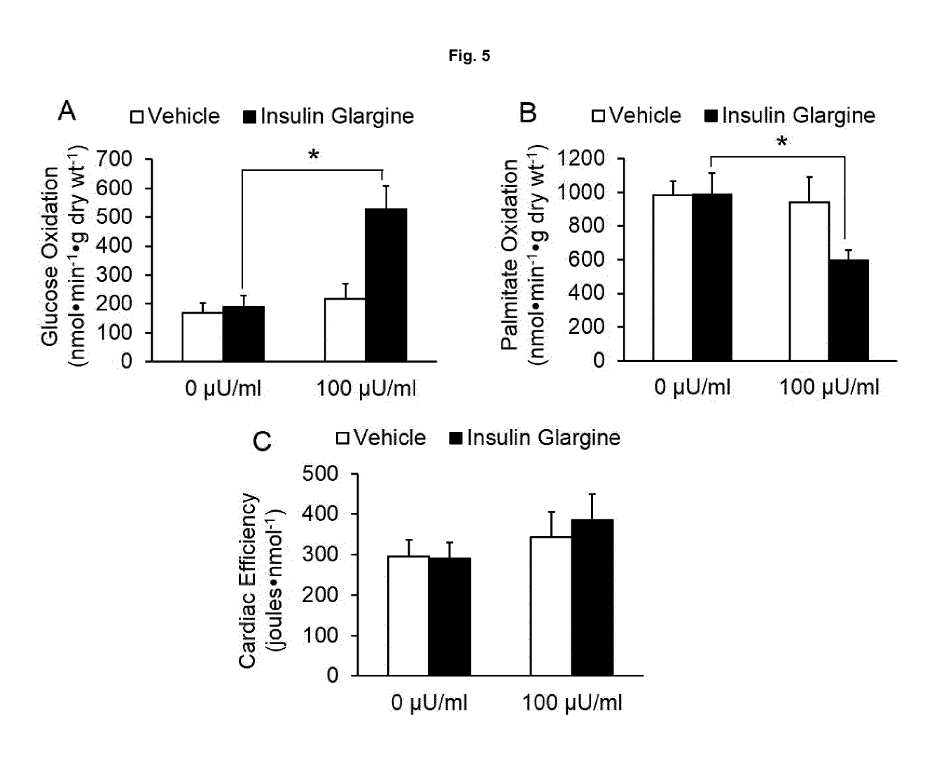

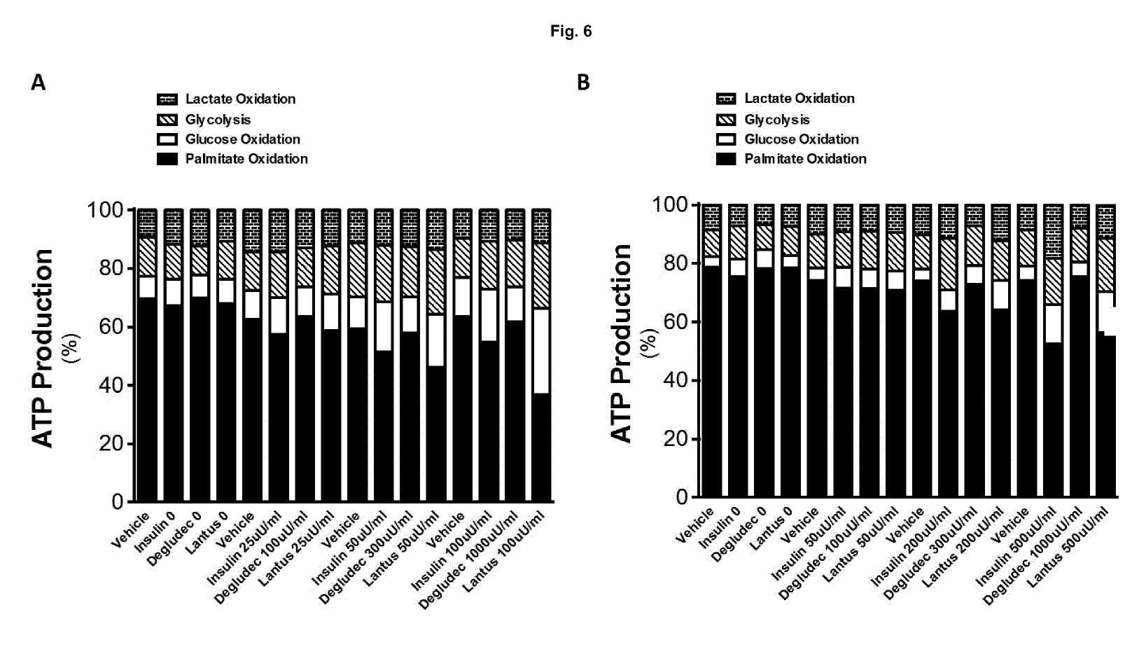

M1-metabolite of insulin glargine, but not insulin Degludec, was able to stimulate glucose oxidation and inhibit palmitate oxidation in both C57bl/6 and db/db mouse hearts (FIGS. 2 to 5). This resulted in an increase in the % ATP from glucose oxidation in both C57bl/6 (vehicle, 13%; insulin, 18%; Degludec, 12%; Lantus, 30%) and db/db (vehicle, 5%; insulin, 13%; Degludec, 5%; Lantus, 14%) mouse hearts (FIG. 6). They also had distinct effects on the % ATP from palmitate oxidation in both C57bl/6 (vehicle, 64%; insulin, 55%; Degludec, 62%; Lantus, 37%) and db/db (vehicle, 74%; insulin, 53%; Degludec, 75%; Lantus, 56%) mouse hearts (FIG. 6).

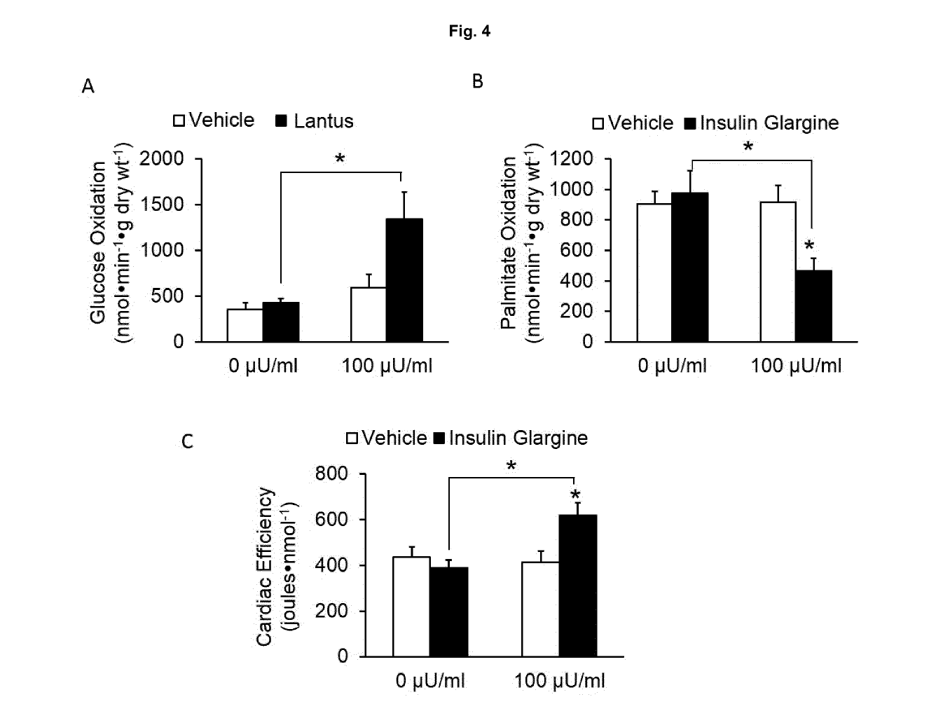

The M1 metabolite of insulin glargine also improved cardiac efficiency of C57bl/6 and db/db mouse hearts (FIG. 4).

### Example 3: Acute Lantus Treatment Stimulates Insulin Signaling More Than Insulin or Degludec

The phosphorylation status of several enzymes involved in insulin signaling were also assessed in the C57bl/6 and db/db mouse hearts treated acutely with insulin, Degludec, or Lantus, via Western Blot Analysis. Frozen heart issue was homogenized for 30 sec in homogenization buffer (50 mM Tris HCl, 10% glycerol, 1 mM EDTA, 0.02% Brij-35, 1 mM DTT, and protease and phosphatase inhibitors (Sigma)). After sitting on ice at least 10 min, tissue homogenates were centrifuged at 10,000×g for 20 min. The supernatant was then stored at −80° C. Protein concentration of supernatant was determined using the Bradford protein assay. After running samples on SDS-polyacrylamide gel electrophoresis, protein was transferred onto nitrocellulose membrane. Primary antibodies included pAkt Ser473 (Cell Signaling 9271), Akt (Cell Signaling 9272), pGSK3α/β Ser21/9 (Cell Signaling 9331), GSK3β (Cell Signaling 9315), pPDH Ser293 (Calbiochem AP1062), and PDH (Cell Signaling 2784). Protein bands were visualized on autoradiography film using enhanced chemiluminescence (Perkin Elmer) and quantified with Image J.

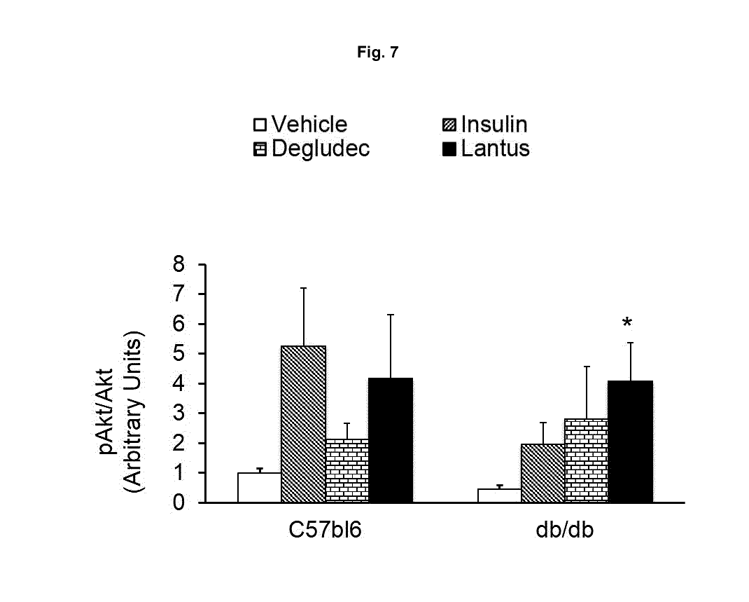

While there was no significant effect on pGSK3β, pPKC, pPDH, or pIRS1 Ser307 (data not shown), Lantus significantly increased pAkt in db/db mouse hearts (FIG. 7).

### Example 4: Chronic Lantus Administration Improves In Vivo Cardiac Function in db/db Mice

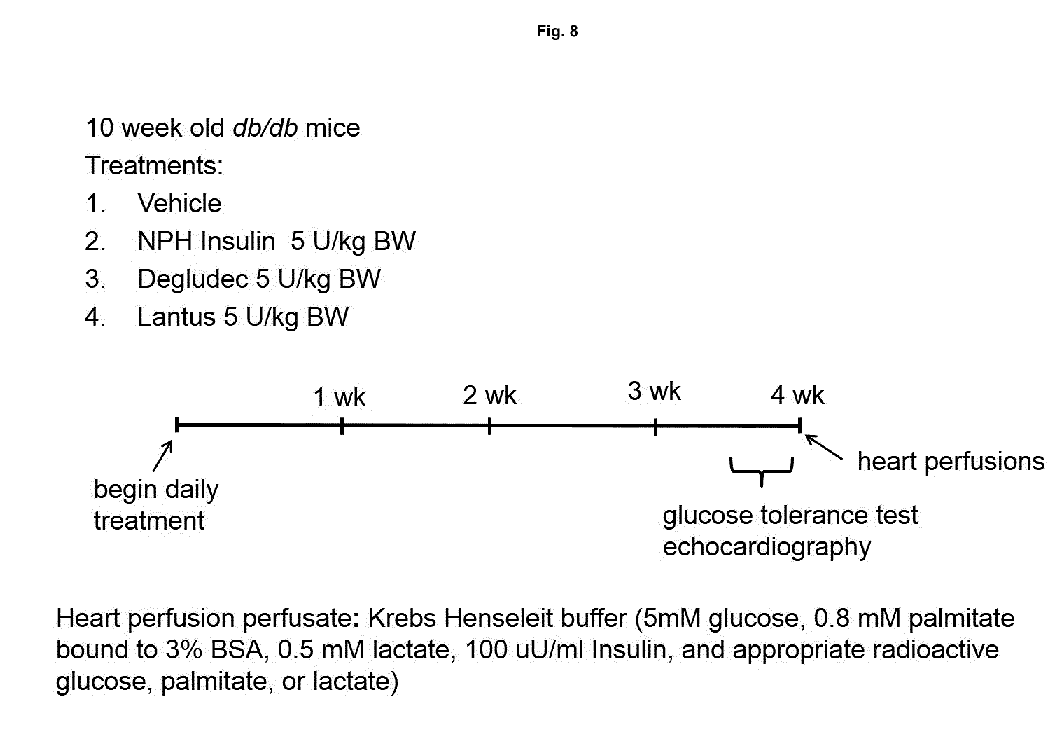

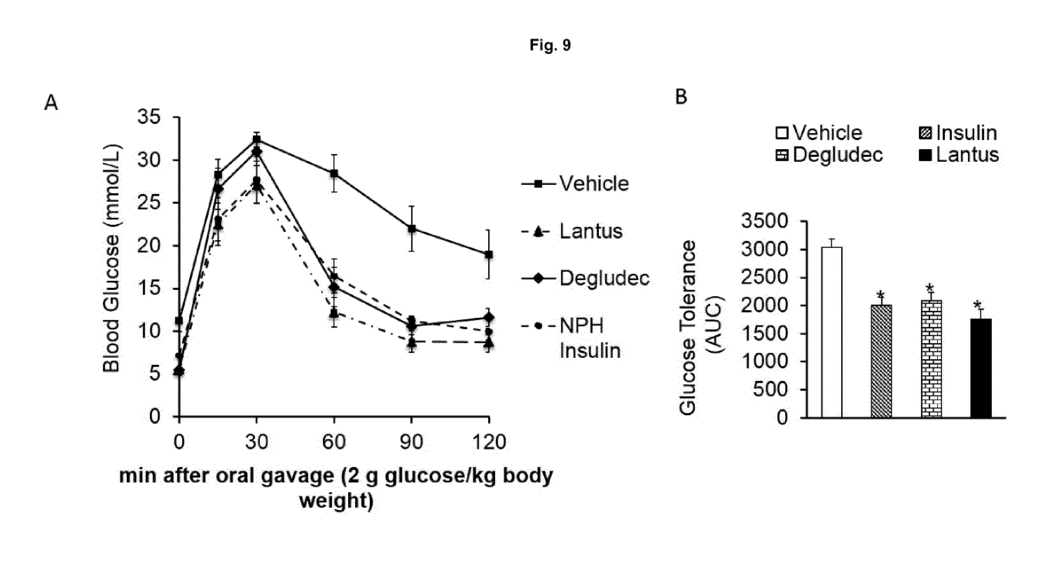

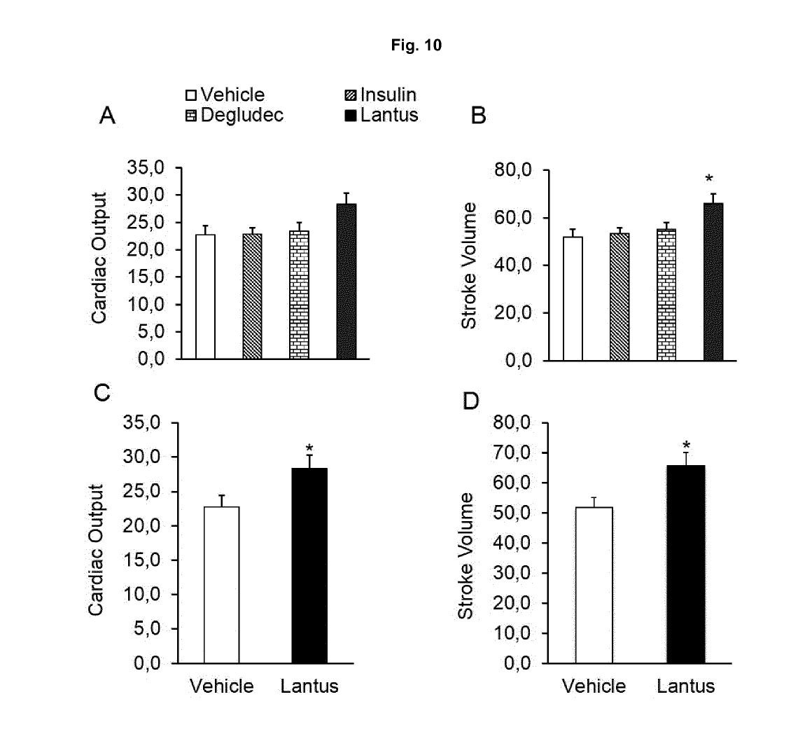

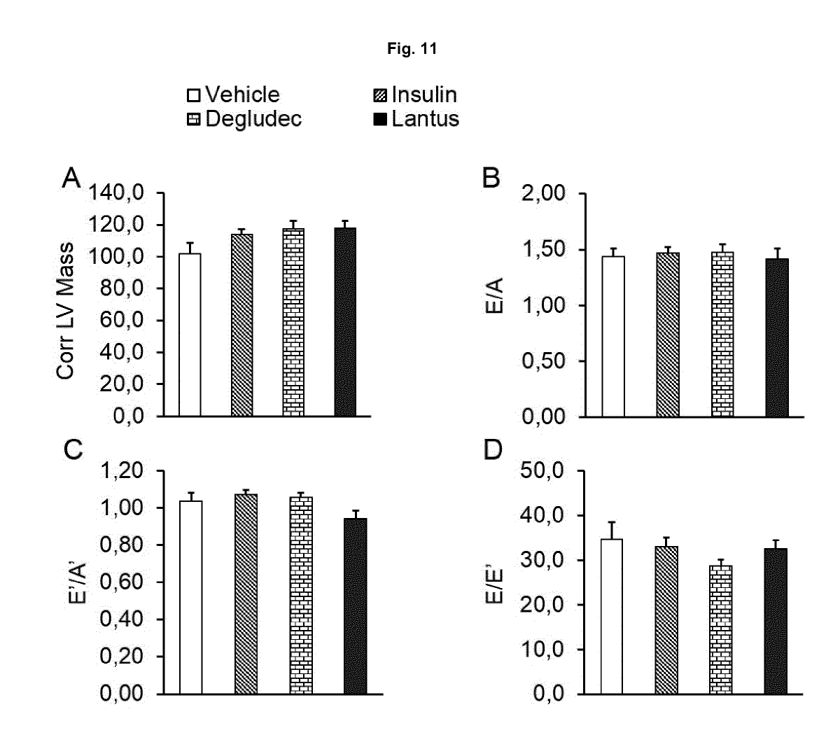

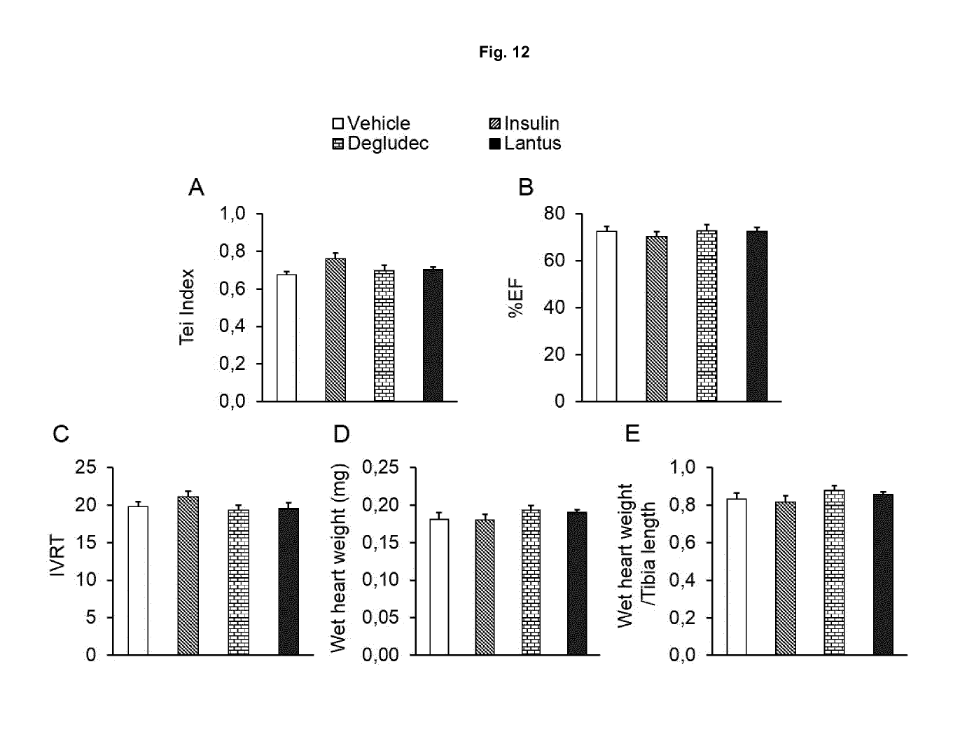

In a second set 18 week old db/db mice were treated with vehicle, NPH insulin, Degludec, or Lantus daily for 4 weeks (see FIG. 8). 18 wk old db/db mice were specifically used because by this age they have a diastolic dysfunction. The original protocol involved treating the mice with either NPH insulin (5 U/kg BW), Degludec (5 U/kg BW), or Lantus (5 U/kg BW). The blood glucose of these mice were tracked and it was noticed that this dose of NPH insulin, Degludec and Lantus did not reduce blood glucose levels (data not shown). Other groups have reported that a higher dose of insulin is required to control hyperglycemia in db/db mice. The highest dose that had been reported to be safe, was 150 U/kg BW, which was then used for future experiments. Treating the mice with 150 U/kg BW of NPH insulin, Degludec, or Lantus improved blood glucose tolerance much more dramatically (FIG. 9). At the end of the 4 week treatment period, the cardiac energy metabolism and function was assessed via echocardiography. The Vevo 770 high resolution echocardiography imaging system was used to assess in vivo cardiac function was assessed in isoflurane anesthetized mice. Results indicated that long acting insulins did not impair db/db mouse cardiac function (FIGS. 11 and 12). Further, chronic treatment of db/db mice specifically with Lantus but not with the other tested insulins, significantly increased cardiac output and cardiac stroke volume (FIG. 10).

### Statistical Analysis

Values are mean±SEM. One-way ANOVA followed by a Bonferonni posthoc test, Kruskal-Wallis test followed by Dunn's Multiple Comparison test, or a t-test were used to determine statistical significance where appropriate. p<0.05 was considered to be significantly different.

In summary, all three tested drugs improved whole body glucose tolerance and did not impair cardiac function or fatty acid and glucose metabolism. However, only Lantus improved cardiac output and stroke volume. Further, only the M1-metabolite of Lantus acutely stimulated glucose oxidation and improved cardiac efficiency in mouse hearts. This increased ability of Lantus to stimulate glucose oxidation may explain why chronic treatment with Lantus improved in vivo cardiac function in db/db mice. Overall, these results indicate that insulin, Degludec and Lantus do not pose a cardiovascular risk in diabetes. Surprisingly, they further show that especially Lantus and its major metabolite may be beneficial, not just for improving glycemic control, but also for reducing cardiovascular dysfunction in diabetic patients.

1. Insulin analogue for use in increasing cardiac output or cardiac stroke volume in a patient at risk of developing or suffering from type I or type II diabetes.

2. Insulin analogue for use in increasing cardiac output or cardiac stroke volume according to claim 1, wherein the cardiac output is increased by 20-30% and/or the cardiac stroke volume is increased by 20-30%.

3. Insulin analogue for use in preventing, delaying and/or treating cardiovascular diseases according to claim 2, wherein the cardiovascular disease is selected from the group consisting of cardiovascular disorders accompanied by decreased cardiac function, including heart failure and related clinical syndromes

4. Insulin analogue for use in increasing cardiac output or cardiac stroke volume or for use in preventing, delaying and/or treating cardiovascular diseases according to any of claims 1 tor 3, wherein the insulin analogue increases the glucose oxidation and/or decreases the fatty acid oxidation in the heart.

5. Insulin analogue for use in increasing cardiac output or cardiac stroke volume or for use in preventing, delaying and/or treating cardiovascular diseases according to any of claims 1 tor 4, wherein the insulin analogue is insulin glargine or glargine-M1.

6. Method of preventing, delaying and/or treating cardiovascular diseases in patients at risk of developing or suffering from diabetes comprising administering a therapeutically effective amount of an insulin analogue.

7. Pharmaceutical composition for use in increasing cardiac output or cardiac stroke volume or for use in preventing, delaying and/or treating cardiovascular diseases comprising an insulin analogue and at least one pharmaceutical acceptable carrier, adjuvant and/or excipient.

8. The pharmaceutical composition for use in increasing cardiac output or cardiac stroke volume or for use in preventing, delaying and/or treating cardiovascular diseases according to any of claim 7, wherein the insulin analogue is insulin glargine or glargine-M1.

9. A method of identifying a patient or of selecting a group of patients who may benefit from treatment with an insulin analogue, wherein said patient or said group of patients is at risk of developing or suffering from type I or type II diabetes, comprising the step of determining whether said patient is at risk of developing or suffering from decreased cardiac output or cardiac stroke volume or cardiovascular disease.

10. The method of claim 9, wherein the cardiovascular disease is selected from the group consisting of cardiovascular disorders accompanied by decreased cardiac function, including heart failure and related clinical syndromes

11. The method of any of claim 9 or 10, wherein the glucose oxidation is increased and/or the fatty acid oxidation in the heart is decreased.

12. The method of any of claims 9 to 11, wherein the cardiac output is decreased by 20-30%.

13. The method of any of claims 9 to 12, wherein the insulin analogue is insulin glargine or glargine-M1.

14. An insulin analogue for use in preventing, delaying and/or treating a cardiovascular disease in a patient at risk of developing or suffering from diabetes, wherein said patient exhibits increased fatty acid oxidation and/or reduced glucose oxidation.

15. A method of indentifying a patient or of selecting a group of patients who will benefit from treatment with an insulin analogue, wherein said patient or said group of patients is at risk of developing or suffering from diabetes and exhibits increased fatty acid oxidation and/or reduced glucose oxidation in the heart.
